(19) United States
(12) Patent Application Publication
(10) Pub. No.: US 2014/0364322 A1
(43) Pub. Date: Dec. 11, 2014

(54) ISOTHERMAL AMPLIFICATION SYSTEMS AND METHODS

(71) Applicants: Mark A. HAYDEN, Ingelside, IL (US); IBIS BIOSCIENCES, INC., Carlsbad, CA (US)

(72) Inventor: Mark A. Hayden, Ingleside, IL (US)

(21) Appl. No.: 14/369,635

(22) PCT Filed: Dec. 28, 2012
(86) PCT No.: PCT/US12/72088

§ 371 Date: Jun. 27, 2014

Related U.S. Application Data
(60) Provisional application No. 61/580,922, filed on Dec. 28, 2011.

(51) Int. Cl. C12Q 1/68

(52) U.S. Cl. CPC C12Q 1/6874; C12Q 1/686
USPC 506/2; 435/289.1; 435/287.2; 435/91.2; 435/6.12; 435/6.11; 506/26; 506/16; 506/9

(57) ABSTRACT

The present invention relates to systems and methods for performing isothermal amplification reactions, in particular, denaturation methods for use in isothermal amplification reactions. An exemplary method may comprise: a) contacting a target nucleic acid with an electrode, wherein the electrode surface has a plurality of first and optionally second nucleic acid primers immobilized thereon, and wherein a target nucleic acid hybridizes to at least one of said first and second nucleic acid primers; b) extending at least one of the first and second primers using a DNA polymerase to form extended target nucleic acids; c) applying positive electrical bias to the electrode such that the extended target nucleic acids anneal to one of the first and second primers; d) extending the target nucleic acid with a DNA polymerase to form amplified target nucleic acid; e) reversing the electrical bias such that the amplified target nucleic acid is denatured from the surface.

The present application claims priority to U.S. Provisional Patent Application No. 61/580,922, filed Dec. 28, 2011, which is herein incorporated by reference in its entirety.

### FIELD OF INVENTION

The present invention relates to systems and methods for performing isothermal amplification reactions. In particular, the present invention relates to denaturation methods for use in isothermal amplification reactions.

### BACKGROUND

Sequencing of nucleic acids continues to be one of the most important and useful ways to analyze DNA and RNA samples. Recent developments have made possible highly parallel high throughput sequencing. Many of these approaches use an in vitro cloning step to generate many copies of each individual molecule. Emulsion PCR is one method, isolating individual DNA molecules along with primer-coated beads in aqueous bubbles within an oil phase. A polymerase chain reaction (PCR) then coats each bead with conal copies of the isolated library molecule and these beads are subsequently immobilized for later sequencing (See, e.g. WO04069849A2 and WO05010145A2). In other cases, surface methods of conal amplification have been developed, for example, by the use of bridge PCR where fragments are amplified upon primers attached to a solid surface. These methods produce many physically isolated locations which each contain many copies of a single fragment. While these methods have provided improvements in sequencing throughput, there is a continuing need to improve the methods of obtaining samples appropriate for sequencing, and of handling, storing, and amplifying such samples. In particular, there is a need to improve methods for obtaining high throughput sequencing data for a specific set of genes or gene products from whole genome or transcriptome samples.

Therefore, there is a need for improved methods of obtaining, storing, amplifying, and analyzing DNA and RNA samples, including methods which can globally or specifically amplify DNA or RNA polynucleotide targets.

### SUMMARY

The present invention relates to systems and methods for performing isothermal amplification reactions. In particular, the present invention relates to denaturation methods for use in isothermal amplification reactions.

Embodiments of the present disclosure provide electronic denaturation methods for use during isothermal amplification. These methods provide the advantage of decreased cost and time savings by eliminating the need for repeated reagent addition, which is required when a chemical denaturant is used.

For example, in some embodiments, the present invention provides a method for amplifying nucleic acid molecules comprising: a) contacting a target nucleic acid with an electrode (e.g., a metal electrode), wherein the electrode surface has a plurality of first and optionally second nucleic acid primers immobilized thereon, and wherein a target nucleic acid hybridizes to at least one of said first and second nucleic acid primers; b) extending at least one of the first and second primers using a DNA polymerase to form extended target nucleic acids; c) applying positive electrical bias to the electrode under conditions such that the extended target nucleic acids anneal to one of the first and second primers; d) extending the extended target nucleic acid with a DNA polymerase to form amplified target nucleic acid; e) reversing the electrical bias such that the amplified target nucleic acid is denatured from the surface; and in some embodiments f) repeating steps a) through c) one or more times. In some embodiments, the electrical bias is delivered via pulses or continuously. In some embodiments, the first and second nucleic acid primers are immobilized to the surface (e.g., via capture nucleic acids). In some embodiments, the method further comprises the step of sequencing the amplified target nucleic acid or identifying a polymorphism or mutation in the amplified target nucleic acid. In some embodiments, the method is performed at an isothermal temperature. In some embodiments, the sequence is determined by incorporation of labeled nucleotides (e.g., a fluorescent group or a radioactive label).

Additional embodiments provide kits and systems comprising reagents necessary, sufficient or useful for performing the aforementioned amplification reactions.

In some embodiments, the present invention provides a system for performing isothermal amplification, comprising: an electrode (e.g., metal electrode) with a plurality of first and optionally second nucleic acid primers immobilized thereon; and b) a device for applying electrical current to the electrode. In some embodiments, the system further comprises one or more of a device for controlling temperature of the electrode, an automated sample handling component, reagents for performing amplification and/or sequencing reactions using the system (e.g., nucleotides, polymerases, buffers, etc), or a detection component (e.g., a charge-coupled-device camera).

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 shows an overview of embodiments of the present invention that utilize bridge amplification.

FIG. 2 shows an overview of a polymerization step in an amplification reaction of embodiments of the present invention.

FIG. 3 shows an overview of a denaturation step in an amplification reaction of embodiments of the present invention.

### DETAILED DESCRIPTION

The present invention relates to systems and methods for performing isothermal amplification reactions. In particular, the present invention relates to denaturation methods for use in isothermal amplification reactions.

### DEFINITIONS

To facilitate an understanding of the present technology, a number of terms and phrases are defined below. Additional definitions are set forth throughout the detailed description.

As used herein, “a” or “an” or “the” can mean one or more than one. For example, “a” widget can mean one widget or a plurality of widgets.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “isothermal amplification” refers to an amplification reaction performed at a single temperature. In some embodiments, the denaturation step is affected by chemical reagents or an electrical charge, rather than by heat.

“Solid support” as used herein refers to any solid surface to which nucleic acids can be attached, such as for example, including but not limited to, metal surfaces, latex beads, dextran beads, polystyrene surfaces, polypropylene surfaces, polyacrylamide gel, gold surfaces, glass surfaces and silicon wafers.

“Means for immobilizing nucleic acids to a solid support” as used herein refers to any chemical or non-chemical attachment method including chemically-modifiable functional groups. “Attachment” relates to immobilization of nucleic acid on solid supports by either a covalent attachment or a non-covalent attachment.

As used herein, the term “bridge amplification” refers to an amplification reaction where one or more of template and primer molecules are immobilized on a support, forming therefore a bridge-like structure when they hybridize.

As used herein, the terms “subject” and “patient” refer to any animal, such as a dog, a cat, a bird, livestock, and particularly a mammal, and preferably a human.

As used herein, the term “sample” is used in its broadest sense. In one sense, it is meant to include a representative portion or culture obtained from any source, including biological and environmental sources. Biological samples may be obtained from animals (including humans) and encompass fluids, solids, tissues, and gases. Biological samples include blood products, such as plasma, serum, and the like. Environmental samples include environmental material such as surface matter, soil, mud, sludge, biofilms, water, and industrial samples. Such examples are not however to be construed as limiting the sample types applicable to the present invention.

### Embodiments of the Technology

Although the disclosure herein refers to certain illustrated embodiments, it is to be understood that these embodiments are presented by way of example and not by way of limitation.

In some embodiments, the present invention provides compositions and methods for use in isothermal PCR (e.g., bridge amplification). Bridge amplification is a method where surface immobilized DNA targets are amplified within clusters using isothermal PCR. In this method, DNA targets and/or primers are fused to capture oligos on the surface and amplified by repeated melting and polymerization using a polymerase and dNTP nucleotides.

During each cycle of an amplification reaction, it is necessary to denature the DNA template from the capture primer sequences on the surface. Traditionally, a chemical denaturant, such as N,N-dimethylformamide (DMF), is repeatedly flushed in at the beginning of every cycle to denature the DNA. High temperature melting cannot be used since the reaction is isothermal. This repeated chemical denaturation step has several disadvantages including long reaction times due to the presence of an additional step in the overall reaction, and high reagent cost from the repeated sequential addition of new amplification reagents after chemical denaturation.

In some embodiments, the present disclosure provides improved methods for denaturing DNA during isothermal PCR. For example, in some embodiments, the chemical denaturation step is replaced with a charge-based denaturation step, which eliminates the need for repeated flushing of reagents at each cycle, thereby reducing the overall reaction time and reagent costs.

In some embodiments, an electric potential is applied to an electrode (e.g., metal electrode) to generate highly localized electrostatic field that is used to denature nucleic acid targets from an electrode surface. Electrostatic dissociation of surface-bound DNA hairpin structures has been described previously (Wei et al., Langmuir 22:6280 (2006); herein incorporated by reference in its entirety).

In Wei et al., discrimination of matched and single-base mismatches in the stem-loop structures were realized by measuring differences in the melting voltage (Vm). In this example, different voltage potentials were used to discriminate thermodynamic differences in the hybridization energies. In a similar concept, another group used alternating reverse fields to enhance or diminish DNA hybridization on a solid surface by utilizing the high negative charge density of immobilized DNA (Wong et al., Nano Lett. 9:3521 (2009)). Using dynamic fields, rather than static fields, enhanced hybridization rates of solution probes to the surface-bound targets were observed.

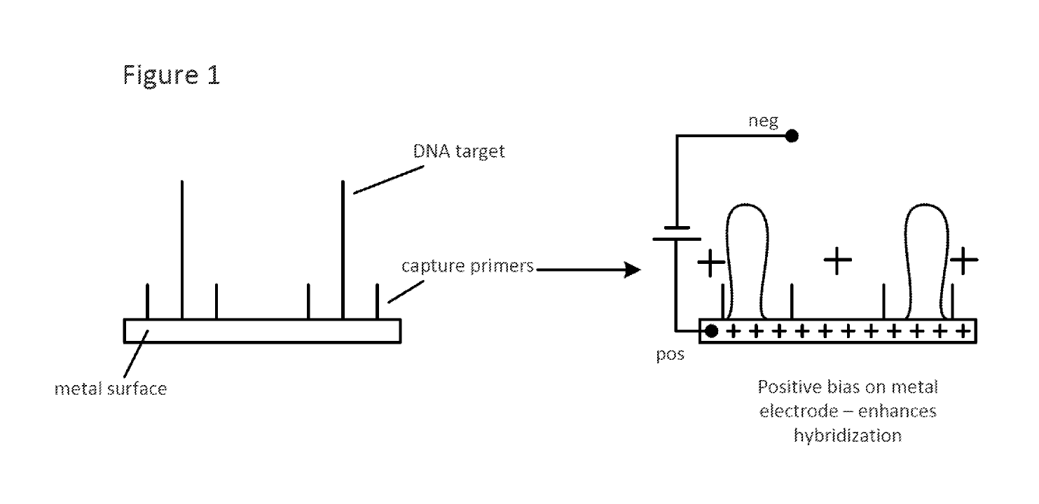

Embodiments of the present invention utilize an alternating electrostatic field in isothermal amplification (e.g., bridge amplification) reactions as a way to reduce reaction time and lower reagent costs. The present invention is exemplified as applied to bridge amplification. However, the methods described herein find use in a variety of amplification reactions and are not limited to application in bridge amplification reactions. In some embodiments, a primer capture monolayer is first placed on a metal electrode surface using standard immobilization chemistry (e.g., thiolated probes on gold, carboxylated silicon surface via ester condensation, etc). The present invention is not limited to a particular capture primer. Exemplary capture primers include but are not limited to, any number of nucleotide-based analogs including standard DNA or RNA oligos, PNA-based probes, or others. Specific DNA or RNA targets of interest are hybridized to the capture primers and extended via polymerase to generate covalently linked targets to the metal surface. Once extended, the complementary ends on the opposite end of the immobilized target are driven to hybridize with surface primers by generating a positive potential bias on the metal surface. This bias causes an electrostatic attraction between the polyanionic nucleic acid strand and the metal surface, which contains the monolayer of capture primers. A schematic of an exemplary application of the methods is shown in FIG. 1.

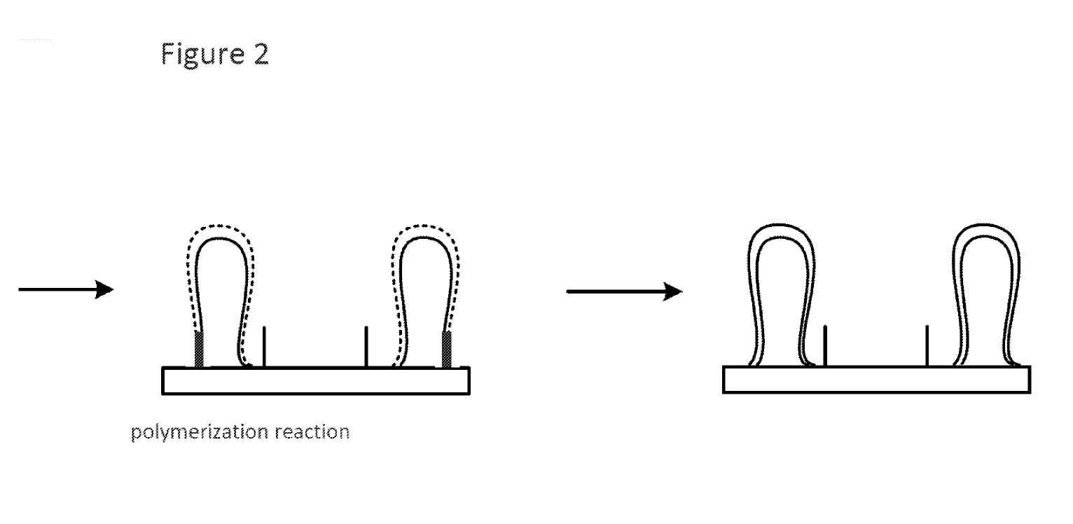

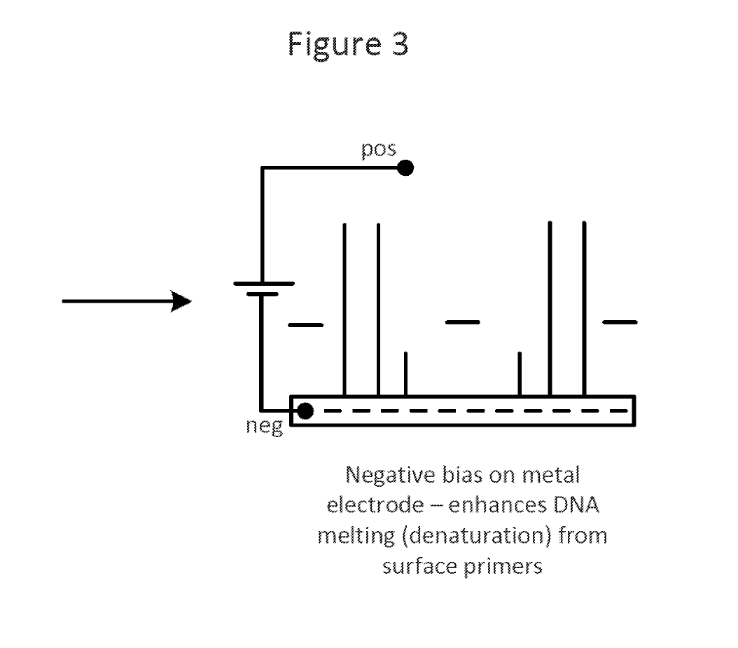

Once the nucleic acid target hybridizes with the surface primers, the target is then extended via the polymerization reagents (e.g., polymerase, dNTP's, buffer, etc.). The polymerized double stranded target is now ready for a second round of denaturation prior to the next round of amplification (FIG. 2). In some embodiments, this denaturation is accomplished by reversing the original electrical field by placing a negative potential bias on the surface electrode. This negative bias causes repulsion of the DNA target from the surface, thereby resulting in a migration of the free ends away from the surface (FIG. 3). Denaturation of the double stranded target then allows for subsequent polymerization cycles, which results in a clonally enriched region or cluster on the surface. The electronic voltage sufficient to cause denaturation is determined empirically and generally depends on the hybridization length, base composition and salt concentration. In some embodiments, electronic potentials are a series of short pulses or longer low voltage settings. This electronic approach eliminates the need for repeated reagent addition, which is required when a chemical denaturant is used. With electronic denaturation, there is no need to remove the polymerization reagents.

The present invention is not limited to a particular isothermal amplification method. In some embodiments, amplification methods are solid-phase amplification, polony amplification, colony amplification, emulsion PCR, bead RCA, surface RCA, surface SDA, etc., as will be recognized by one of skill in the art. In some embodiments, amplification methods that results in amplification of free DNA molecules in solution or tethered to a suitable matrix by only one end of the DNA molecule are used. In some embodiments, methods that rely on bridge PCR, where both PCR primers are attached to a surface (see, e.g., WO 2000/018957, U.S. Pat. Nos. 7,972,820; 7,790,418 and Adessi et al., Nucleic Acids Research (2000): 28(20): E87; each of which are herein incorporated by reference) are used. In some cases the methods of the invention can create a “polymerase colony technology”, or “polony”, referring to a multiplex amplification that maintains spatial clustering of identical amplicons (see Harvard Molecular Technology Group and Lipper Center for Computational Genetics website). These include, for example, in situ polonies (Mitra and Church, Nucleic Acid Research 27, e34, Dec. 15, 1999), in situ rolling circle amplification (RCA) (Lizardi et al., Nature Genetics 19, 225, July 1998), bridge PCR (U.S. Pat. No. 5,641,658), picotiter PCR (Leamon et al., Electrophoresis 24, 3769, November 2003), and emulsion PCR (Dressman et al., PNAS 100, 8817, Jul. 22, 2003).

In some embodiments, primers for bridge amplification are, along with the nucleic acid template, modified to aid in immobilization on a solid support. In some embodiments, nucleic acids are immobilized using only the 5′ end, which leaves the 3′ end remote from the support such that the 3′ region of templates and primer can hybidize and drive the synthesis of a new strand.

The immobilization protocol preferably avoids the release of templates and primers from the solid surface during the isothermal amplification. Therefore, in some embodiments, the means for immobilization are chemically modifiable functional groups that allow the formation of a covalent binding between said molecules and a solid support having a derivatised surface, which is subsequently modified with bifunctional crosslinking groups to provide a functionalized surface. Alternatively, the immobilization can be obtained by irreversible passive adsorption or by exploiting the affinity between specific molecules (for example, immobilization on an avidin-coated surface by biotinylated molecules).

Examples of chemically modifiable functional groups to be added at the 5′ end of the nucleic acids to be immobilized include, but are not limited to, thiol, hydroxyl, dimethoxyltrityl (DMT), amino, or phosphate groups, as well as carboxylic or aldehyde moieties. Examples of crosslinking agents useful to derivatise a solid support are 1-ethyl-3-(3-dimethylaminopropyl)-carbodiimide hydrochloride (EDC), succinic anhydride, phenyldiisothiocyanate or maleic anhydride, or a hetero-bifunctional crosslinker such as for example m-maleimidobenzoyl-N-hydroxysuccinimide ester (MBS), N-succinimidyl[4-iodoacethyl]aminobenzoate (SIAB), Succinimidyl 4-[N-maleimidomethyl]cyclohexane-1-carboxylate (SMCC), N-y-maleimidobutyryloxy-succinimide ester (GMBS), Succinimidyl-4-[p-maleimidophenyl]butyrate (SMPB) and the sulfo (water-soluble) corresponding compounds. In some embodiments, the nucleic acid templates and primers are modified with thiol, phosphate or amino group at the 5′ end modification and immobilized using an immobilization solution containing 1-ethyl-3-(3-dimethylaminopropyl)-carbodiimide hydrochloride (EDC) as crosslinking agent.

Although the solid support may be any solid surface to which nucleic acids can be immobilized, such as for example latex or dextran beads, polystyrene surfaces, polypropylene surfaces, polyacrylamide gel, gold surfaces, glass surfaces and silicon wafers, preferably the solid support is rigid enough to be not affected by the force deployed during the elongation and allowing a covalent attachment of the nucleic acid through functionalization using mono- or bifunctional crosslinking reagents. In some embodiments, the solid support is metal or other material that conducts electricity.

The size and the number of DNA colonies can be controlled by modulating the time during which the solid support is subjected to the isothermal amplification. Thus the number of nucleic acid colonies formed on the surface of the solid support is dependent upon the number of nucleic acid templates which are initially immobilized to the support, provided there is a sufficient number of immobilized colony primers at a distance allowing the hybridization with template molecules. By controlling the initial density of the nucleic acid templates and primers, an optimum situation can be reached wherein a high density of individual nucleic acid colonies can be produced isothermally on a solid support of a size sufficient to allow their analysis and containing a large enough number of amplified sequences.

The isothermal amplification can be performed using a DNA- or a RNA-dependent DNA polymerase, plus a supply of nucleoside triphosphate molecules or any other nucleotide precursors, for example modified nucleoside triphosphate molecules.

Examples of nucleic acid polymerases suitable for use in embodiments of the present invention include, but are not limited to, DNA polymerase (Klenow fragment, T4 DNA polymerase), thermostable DNA polymerases (Perler F. B. et al., Adv. Protein Chem. 1996, 48:377-435) identified and cloned in a variety of thermostable bacteria (such as Taq, VENT, Pfu, Tfl DNA polymerases) as well as their genetically modified derivatives (TaqGold, VENTexo, Pfu exo). Preferably the nucleic acid polymerase used for colony primer extension is stable under temperature at which the primer and template hybridization results enough specific to avoid incomplete or spurious amplifications of the template.

The amplification solution contains preferably, as nucleotide precursors, deoxyribonucleotide triphosphates, for example dATP, dTTP, dCTP, dGTP, naturally or non-naturally occurring, for example modified with a fluorescent or radioactive group. A large variety of synthetically modified nucleic acids have been developed for chemical and biological methods in order to increase the detectability and/or the functional diversity of nucleic acids. These functionalized/modified molecules can be fully compatible with natural polymerizing enzymes, maintaining the base pairing and replication properties of the natural counterparts, as recently reviewed (Thum O et al., Angew. Chem. Int. Ed. 2001, 40 (21): 3990-3993).

When the template is either a RNA or a DNA molecule and it is desired to obtain amplification products in the form of double stranded RNA, the nucleic acid polymerase can be a RNA- or a DNA-dependent RNA polymerase, for example a viral RNA replicase or a ribozyme (Johnston W K et al., Science 2001, 292(5520):1319-25; Tayon R Jr et al., Nucleic Acids Res 2001, 29(17):3576-82). The amplification solution will consequently contain, as nucleotide precursors, ribonucleotide triphosphates.

Other components of the amplification solution are added consequently to the choice of the nucleic acid polymerase, and they are essentially corresponding to compounds known in the art as being effective to support the activity of each polymerase. The concentration of compounds like dimethyl sulfoxide (DMSO), Bovine Serum Albumin (BSA), Triton X-100, or MgCl₂ is well known in the prior art as being important to have an optimal amplification, and therefore the operator can easily adjust such concentrations for the methods of the present invention on the basis of the examples presented hereafter.

Nucleic acids which may be amplified according to the methods of the invention include DNA, for example, genomic DNA, complementary DNA, recombinant DNA or any form of synthetic or modified DNA. Their length may vary amongst template molecules simultaneously immobilized on the same solid support, and they may be fragments or smaller parts of larger nucleic acid molecules having a known or unknown sequence. The nucleic acids to be amplified may be derived from any source (e.g., genomic DNA fragments obtained by limited restriction enzyme digestion). In the case of mRNA, the isolated mRNA can be transformed into cDNA by the use of a reverse transcriptase and, eventually, into double stranded DNA before use

Alternatively, whenever the template is a natural, synthetic, or modified RNA molecule, it can be directly amplified by the method of the invention using specific enzyme known in the art that can generate double stranded RNA starting from single stranded RNA.

When producing the nucleic acid templates and primers additional desirable sequences can be introduced by methods well known and documented in the art. Such additional sequences include, for example, restriction enzyme sites, modified nucleotides, or other nucleic acid tags enabling the identification and/isolation of amplification products containing a given nucleic acid template sequence. Other desirable sequences include fold-back DNA sequences (which form hairpin loops or other secondary structures when rendered single-stranded), ‘control’ DNA sequences which direct protein/DNA interactions, such as a promoter, an enhancer, a replication origin, or an operator DNA sequence which are recognized by specific DNA-binding proteins.

The present invention is not limited to a particular amplification technique. The electronic denaturation methods described herein find use in a variety of applications that utilize amplification (e.g., microarrays, SNP detection and nucleic acid sequencing).

In some embodiments, the systems and methods described herein find use in nucleic acid sequence methods. In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In some embodiments, nucleic acids amplified using the methods described herein find use in are variety of research, screening and clinical applications, including but not limited to, in the fields of genomics, pharmacogenomics, drug discovery, food characterization, genotyping, diagnostics, gene expression monitoring, genetic diversity profiling, whole genome sequencing and polymorphism discovery, or any other applications involving the amplification of nucleic acids.

A yet further aspect of the invention provides kit and systems comprising components necessary, sufficient or useful for performing isothermal nucleic acid amplification using electrical denaturation (e.g., nucleic acids, solid supports, reagents, controls, etc.).

In some embodiments, the present invention provides systems and devices for performing isothermal amplification (e.g., as an aspect of a nucleic acid sequencing reaction). Such an apparatus can include one or more of the following: a) means for immobilizing primers on a surface (although this is not needed if immobilized primers are already provided); b) a supply of a nucleic acid polymerase; c) a supply of precursors of the nucleotides to be incorporated into a nucleic acid (e.g. a supply of nucleoside triphosphates); d) electrode for separating annealed nucleic acids (e.g. metal electrode); e) a means for controlling temperature and electric current through electrode; f) a detection component for detecting amplified nucleic acids; and g) control means for coordinating the different steps required for the method of the present invention.

Other apparatuses are within the scope of the present invention. These allow immobilized nucleic acids to be analyzed. They can include a source of reactants and detecting means for detecting a signal that may be generated once one or more reactants have been applied to the immobilized nucleic acid molecules. In some embodiments, the device for detecting a signal has sufficient resolution to enable it to distinguish between signals generated from different colonies.

Various detection systems can be used to detect labels used in sequencing methods (although in certain embodiments detection may be possible simply by eye, so that no detection system is needed). One detection system for fluorescent labels is a Charge-Coupled-Device (CCD) camera, which can optionally be coupled to a magnifying device. Any other device allowing detection and, preferably, also quantification of fluorescence on a surface may be used. Devices such as fluorescent imagers or confocal microscopes may be chosen.

In some embodiments, apparatuses are preferably provided in automated form so that once they are activated, individual process steps can be repeated automatically.

For example, in some embodiments, systems include an electrode (e.g., metal electrode) for performing isothermal bridge amplification, along with devices for reading the results of sequencing reactions. In some embodiments, electrodes (e.g., metal electrodes) are incorporated into existing platforms for bridge amplification and next generation sequencing (e.g., those described herein).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### Example

A flow cell is designed with two separate electrodes positioned on the surface of the flow cell and above the cell. The electrode material may be composed of any metal or alloy that is capable of conducting an electrical current. In this example, gold is used as the metal electrode material. Application of a DC current causes the solution in the flow cell to be exposed to an electrical potential with the surface electrode either positively charged or negatively charged, depending on the bias polarity. The electrical potential is used to enhance surface DNA hybridization or denaturation.

Initially, a series of capture probes are deposited on the gold surface for the purpose of hybridizing nucleic acid targets to be sequenced. The probes are composed of short (15-50 bases) oligonucleotides labeled on the 5′ and/or 3′ end with a mercaptan group so that they can be attached to the gold surface. The capture probes are at a specific concentration needed to ensure proper spacing on the gold surface for subsequent clonal amplification via bridge amplification. After attachment of the capture probes, the remaining gold surface is passivated using a standard passivation reagent, such as octanethiol. The passivation process establishes a self-assembled monolayer (SAM) of octanethiol on the gold surface with “islands” of capture probes at a predetermined density.

A single-stranded target nucleic acid sequence is added to the flow cell in an appropriate hybridization buffer and allowed to hybridize to under standard conditions. These conditions will vary depending on the G/C content and length of the complementary sequence overlap. Typically, hybridization buffers consist of 10-500 mM salt with 10-500 mM Tris buffer at pH 6-9. Hybridization temperature can vary as well, ranging from 20° to 75° C., depending on the sequence. The target nucleic acid is allowed to cross-hybridize to the capture probes on the surface to form the “bridge” for bridge amplification.

Once the hybridized structure is formed, the hybridization solution is washed out and replaced with the amplification reagents, consisting of a DNA polymerase, deoxynucleotides, enzymatic cofactors (such as Mg⁺²), and buffer. The polymerization reaction is allowed to proceed, which produces one copy of the target sequence. Denaturation of the surface-bound target is accomplished by application of a small electrical potential ranging from 1-1,000 mV, which causes the surface electrode to have a negative charge, thereby repelling the target from the surface-bound capture probes. This results in a denatured state for the target nucleic acid. A second electrical bias pulse is applied in the reverse polarity, which causes the open end of the target to be attracted to the positively charged gold surface, thereby enhancing hybridization. This is followed by a second round of amplification. The entire process is repeated with alternating cycles of reverse polarity until clonal clusters are generated on the surface. The clusters are now ready for the subsequent sequencing reaction.

We claim:

1. A system for amplifying nucleic acids at an isothermal temperature, comprising:
a) an electrode with a plurality of first nucleic acid primers immobilized thereon;
b) a device for applying an electrical potential to said electrode.

2. The system of claim 1, wherein said electrode is a metal electrode.

3. The system of claim 1, further comprising a plurality of second nucleic acid primers immobilized thereon.

4. The system of claim 1, further comprising a device for controlling temperature of said electrode.

5. The system of claim 1, further comprising an automated sample handling component.

6. The system of claim 1, wherein said system further comprises reagent for performing amplification and/or sequencing reactions using said system.

7. The system of claim 1, wherein said system further comprises a detection component.

8. The system of claim 7, wherein said detection component is a charge-coupled-device camera.

9. A method of performing isothermal amplification of nucleic acid molecules, comprising:
a) applying a target nucleic acid to the proximity of an electrode, wherein said electrode surface has a plurality of first nucleic acid primers immobilized thereon, and wherein said target nucleic acid hybridizes to said first nucleic acid primers;
b) amplifying said target nucleic acid, wherein said amplifying comprises extension and denaturation steps, wherein said denaturation is charge based denaturation.

10. The method of claim 9, wherein said amplification comprises one of more of the steps of:
a) extending said first primer using a DNA polymerase to form extended target nucleic acids;
b) applying positive electrical bias to said electrode under conditions such that said extended target nucleic acids anneal to said first primer;
c) extending said extended target nucleic acid with a DNA polymerase to form amplified target nucleic acid; and
d) reversing said electrical bias such that said amplified target nucleic acid is denatured from said surface.

11. The method of claim 9, further comprising the step of repeating steps a) through b) one or more times.

12. The method of claim 10, wherein said electrical bias is delivered via pulses or continuously.

13. The method of claim 9, further comprising a plurality of second nucleic acid primers.

14. The method of claim 13, wherein said first and second nucleic acid primers are immobilized to said metal surface via capture nucleic acids.

15. The method of claim 10, further comprising the step of sequencing said amplified target nucleic acid.

16. The method of claim 9, further comprising the step of identifying a polymorphism or mutation in said amplified target nucleic acid.

17. The method of claim 16, wherein the sequence is determined by incorporation of labeled nucleotides.

18. The method of claim 17, wherein the label is a fluorescent group or a radioactive label.
